(19) United States
(12) Patent Application Publication
(10) Pub. No.: US 2011/0307036 A1
(43) Pub. Date: Dec. 15, 2011

(54) COMPOSITIONS AND METHODS FOR ENHANCING COLLAGEN GROWTH

(76) Inventor: Chien-Min Sung, Tansui (TW)

(21) Appl. No.: 13/115,328

(22) Filed: May 25, 2011

Related U.S. Application Data
(60) Provisional application No. 61/354,109, filed on Jun. 11, 2010.

(51) Int. Cl. A61N 5/06

(52) U.S. Cl. 607/95

(57) ABSTRACT

The present invention provides methods for enhancing collagen growth in a subject's skin. In one aspect, for example, such method can include disposing a composition including a plurality of nanodiamond particles dispersed in a cosmetically acceptable carrier onto a subject's skin, applying energy to the plurality of nanodiamond particles such that at least a portion of the energy is absorbed by the nanodiamond particles, and emitting the energy from the nanodiamond particles as IR radiation, and delivering the IR radiation to the skin in order to heat the skin and enhance collagen growth.

### PRIORITY DATA

This application claims the benefit of U.S. Provisional Patent Application Ser. No. 61/354,109, filed on Jun. 11, 2010, which is incorporated herein by reference.

### FIELD OF THE INVENTION

The present invention relates to compositions and to methods for enchancing the growth of collagen in a subject. Accordingly, the present invention involves the fields of chemistry and healthcare.

### BACKGROUND OF THE INVENTION

The human skin comprises several layers, including epidermis (cuticle), dermis (cornium), and the hypodermis (subcutaneous tissue). The dermis layer is an elastic skin layer, which is composed primarily of collagen and elastin proteins. It contains, among other things, a dense network of collagen fibres, which are filled with elastic connective tissue.

Collagen is one of the predominant proteins in connective tissue, making up about 25% to 35% of whole-body protein content. There are more than 25 types of collagens that naturally occur in the body. Collagen proteins possess good tensile strength, and are important in contributing to the external support of cells, and provide firmness and strength to the body, including the skin. Particularly relating to the skin, collagen, in combination with keratin, provides strength, flexibility, and resilience to the skin.

As people age, however, collagen degradation occurs, often leading to wrinkles It is, therefore, an important substance for those looking for ways to fight the visible effects of aging on the skin.

### SUMMARY OF THE INVENTION

The present invention provides methods for enhancing collagen growth in a subject's skin. In one aspect, for example, such method can include disposing a composition including a plurality of nanodiamond particles dispersed in a cosmetically acceptable carrier onto a subject's skin, applying energy to the plurality of nanodiamond particles such that at least a portion of the energy is absorbed by the nanodiamond particles, and emitting the energy from the nanodiamond particles as IR radiation, and delivering the IR radiation to the skin in order to heat the skin and enhance collagen growth.

Various forms of energy can be utilized for absorption into the plurality of nanodiamond particles, and any form of energy that can be absorbed and safely emitted as IR radiation should be included within the present scope. In one aspect, for example, the energy is sunlight. In another aspect, the energy is UV radiation. In a more specific aspect, the UV radiation is delivered via a UV light source. In yet another aspect, the energy can be laser radiation.

The emitted IR radiation can enhance collagen growth by increasing the temperature underneath the epidermal skin of a subject. The degree of heating can vary depending on physiological differences between individuals, personal preferences, and the like. In one aspect, the IR radiation increases temperature underneath epidermal skin by from about 0.5° C. to about 3.0° C. In another aspect, the IR radiation increases temperature underneath epidermal skin by from about 1.5° C. to about 2.5° C.

A number of cosmetically acceptable carriers are contemplated, non-limiting examples of which can include water, gels, glycerin, alcohols, emollients, fatty acids, fatty alcohols, maltodextrin, carrageenans, microcrystalline cellulose (MCC) or other celluloses, sugars, alcohol sugars, lactose, and combinations thereof. In some aspects the cosemetically acceptable carrier can include a dispersant to disperse the nanodiamond particles therein. While any dispersant capable of dispersing nanodiamond particles in a given carrier should be considered to be within the present scope, non-limiting examples can include anionic surfactants, electrolytes, alcohols, metal chlorides, metal nitrates, viscous biologically acceptable carriers, and combinations thereof.

The nanodiamond particles of the present application can be utilized in various sizes and proportions. In one aspect, for example, the nanodiamond particles have an average size of from about 5 nm to about 900 nm. In an additional aspect, the average nanodiamond particle size may be from about 2 nm to about 500 nm. In another aspect the nanodiamond particles have an average size from about 2 nm to about 50 nm. In yet another aspect the nanodiamond particles have an average size of less than or equal to about 10 nm.

Furthermore, in one aspect the nanodiamond particles comprise from about 1 wt % to about 60 wt % of the composition. In another aspect the nanodiamond particles comprise from about 1 wt % to about 20 wt % of the composition. In yet another aspect the nanodiamond particles comprise less than or equal to about 5 wt % of the composition.

Various functional groups are contemplated for functionalizing the nanodiamond particles. It should be noted that any functional group providing a beneficial effect and that is capable of being bonded to a nanodiamond particle should be considered to be within the present scope. Non-limiting examples include amino, carboxyl, hydroxyl, carbonyl, pyridine, and combinations thereof.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

Definitions

In describing and claiming the present invention, the following terminology will be used in accordance with the definitions set forth below.

The singular forms “a,” “an,” and “the” include plural referents unless the context clearly dictates otherwise. Thus, for example, reference to “a particle” includes reference to one or more of such particles, and reference to “the dispersant” includes reference to one or more of such dispersants.

As used herein, “subject” refers to a mammal that may benefit from the administration of a composition or method of this invention. Examples of subjects include humans, and may also include other animals such as horses, pigs, cattle, dogs, cats, rabbits, and the like, including aquatic mammals.

As used herein, “formulation” and “composition” may be used interchangeably and refer to a combination of elements that is presented together for a given purpose. Such terms are well known to those of ordinary skill in the art.

As used herein, “carrier” and “acceptable carrier” can be used interchangeably and refer to a carrier that may be combined with a plurality of nanodiamond particles in order to provide a desired composition. Those of ordinary skill in the art will recognize a number of carriers that are well known for making specific compositions for administration to tissue.

As used herein, “cosmetically acceptable carrier” refers to a material that is suitable for application to generally external tissue, including skin and keratinous surfaces or other areas of the body. Upon application, cosmetically acceptable carriers are substantially free of adverse reactions with skin and other tissue.

As used herein, the term “cosmeceutical” refers to cosmetic products that impart medicinal or health benefits, as with nutraceuticals. Typically, cosmeceuticals are applied to the skin.

As used herein, the term “topical,” in reference to administration, means applying an active ingredient directly to the skin surface. The active ingredient may be in the form of a composition, to aid in application. Examples of topical formulations include but are not limited to lotions, ointments, creams, gels, sprays, pastes, and powders.

“Skin,” “skin surface,” “derma,” “epidermis,” and similar terms are used interchangeably herein, and refer to not only the outer skin of a subject comprising the epidermis, but also to mucosal surfaces to which a composition may be administered. Examples of mucosal surfaces include the mucosal of the respiratory (including nasal and pulmonary), oral (mouth and buccal), vaginal, introital, labial, and rectal surfaces.

As used herein, “nanoparticle” refers to a nano-sized particle comprising substantially carbon and/or boron nitride. In one aspect, the nanoparticles may be diamond.

As used herein, “remedial” is an adjective referring to remedying, correcting, treating, improving, or preventing an undesirable condition. A remedial composition can therefore be formulated to remove undesirable materials such as sebum, dead skin, and the like from the skin. Similarly, remedial compositions can be configured to remove, prevent or minimize formation of undesirable elements such as odor-producing bacteria and the like.

As used herein, “bonded” and “bonding,” when used in connection with nanodiamond contact with biological materials, refers to bonding such as covalent bonding, ionic bonding, mechanical bonding, van der Waals attractions, hydrogen bonding, or other intermolecular attractive forces.

“Effective amount” refers to an amount of a substance which is sufficient to achieve its intended purpose or effect. Various biological factors may affect the ability of a delivered substance, such as nanodiamond particles, to perform its intended task. Therefore, an “effective amount” may be dependent on such biological factors.

As used herein, “functionalized nanodiamonds” are those nanodiamond particles having surfaces with attached functional groups. The functional groups can absorb or reflect UV radiation, and can be bonded to the nanodiamond particles with a variety of covalent linkages and non-covalent bonding mechanisms. A non-limiting list of covalent linkages includes amines, olefins, thiols, disulfides, and the like. Also, functional groups can be covalently bonded to dangling (i.e. unpaired or otherwise available) electrons on the nanodiamond surface. Thus the purposeful attachment and surface functionalization of nanodiamond particles can include any known means for functionalizing a diamond surface. Potential methods are discussed in U.S. application Ser. No. 11/897,010, filed Aug. 27, 2007, which is incorporated herein by reference.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The Invention

It has been discovered that nanodiamond materials are good black body radiators. In addition to the small size of nanodiamond particles, in detonation nanodiamond the surfaces are often coated with amorphous carbon and numerous organic radicals such as, for example, amines, carboxyls, hydroxides, and the like. Specific examples can include NO₂, CO₂, OH, and the like. Additionally, defects (e.g. microcracks, voids, etc.) and non-bonding carbon (e.g. sp2, sp1) are often present inside the nanodiamond material due to the explosive manufacturing process. All of these radicals and defects can allow the absorption of wide bands of electromagnetic radiation from deep UV to far IR. Nanodiamond particles can radiate these absorbed energies as black body radiation. For example, nanodiamond particles dispersed in water can absorb wavelengths of energy from UV-C through the visible light range, and then emit energy in the far IR (e.g. 8-10 microns in wavelength).

Thus by applying a composition containing nanodiamond particles to the skin of a subject, and subsequently delivering energy such as electromagnetic radiation to the composition, the skin can be heated with IR radiation. This long wavelength IR radiation can penetrate deeply into the skin, from about 1 mm to about 10 mm. As a result, the capillaries of the dermis underlying the epidermis can be increased in temperature by a few degrees centigrade. This increased temperature can enhance the growth of collagen in the skin, thus improving the quality of the skin. Additionally, the radiating properties of the nanodiamond particles can allow even and sustained warmth to permeate the skin.

Additional information regarding nanodiamond compositions can be found in U.S. Pat. No. 7,294,340, filed on Mar. 30, 2004, in U.S. application Ser. No. 11/640,136, filed on Dec. 14, 2006, and in U.S. Provisional Application No. 61/317,135, filed Mar. 24, 2010, each of which are incorporated herein by reference.

Accordingly, the present invention provides methods for improving the quality of skin. In one aspect, for example, a method of enhancing collagen growth in a subject's skin is provided. It should be noted that the scope of the term “enhancing” is intended to include a variety of effects to collagen growth, including “increasing,” “accelerating,” “initiating,” “maximizing,” “moderating,” “facilitating,” and the like. Such effects can be exclusive or nonexclusive, depending on the context. Such a method can include disposing a composition that includes a plurality of nanodiamond particles dispersed in a cosmetically acceptable carrier onto a subject's skin, applying energy to the plurality of nanodiamond particles such that at least a portion of the energy is absorbed by the nanodiamond particles, and wherein the absorbed energy is emitted by the nanodiamond particles as IR radiation. In one aspect, the IR radiation emitted from the nanodiamond has a wavelength of from about 0.5 microns to about 250 microns. In another aspect, the wavelength may be from about 0.7 microns to about 100 microns. In yet another aspect, the wavelength may be from about 0.5 microns to about 10 microns. The IR radiation is delivered to the skin in order to heat the skin and enhance collagen growth. In one aspect, the IR radiation increases temperature underneath epidermal skin by from about 0.5° C. to about 3.0° C. In another aspect, the IR radiation increases temperature underneath epidermal skin by from about 0.5° C. to about 1.5° C. In another aspect, the temperature increase may be from about 1.5° C. to about 3.0° C. In a further aspect, the temperature increase may be from about 1.5° C. to about 2.5° C. Such increases may in some aspects be over and above the average normal temperature for such physiological locations. It will be recognized that in some cases, the average normal temperature may vary from individual to individual, and therefore, in some aspects, the temperature increase may be over and above the average normal temperature for the average individual.

A variety of energy sources can be applied to and absorbed by the nanodiamond particles. It should be noted that any energy source capable of being absorbed and subsequently emitted or radiated by the nanodiamond particles is considered to be within the present scope. As has been described, the energy can be absorbed by the nanodiamond particles and emitted or radiated at a different wavelength. Non-limiting examples of energy sources include sunlight, UV radiation, UV radiation delivered via a UV light source, and the like.

In one aspect, a composition according to aspects of the present invention can include a cosmetically acceptable carrier having nanodiamond particles dispersed therein. The application of such a composition to an area of skin, followed by the application of energy to the area, can cause the temperature underneath the epidermal skin to increase, thus promoting the growth of collagen. Various compositions are contemplated, including liquid emulsions, cream emulsions, powders, dispersions, creams, gels, suspensions, sticks, lotions, water-based dispersions, aerosols, and the like. Lotions can include skin lotions, facial lotions, moisturizers, liquid foundations, eye creams, facial masks (even those that are designed to dry while on the skin), cover up, sunscreens, moisturizers, or any formulation with the consistency matching those products listed. It should also be noted that formulations can be oil based or water based. In some cases, the nanodiamond can be functionalized to be more easily dispersed in a water or oil formulation. For example, in the case of water based formulations, nanodiamonds can benefit from OH, O, and/or N surface terminations. In the case of oil based formulations, nanodiamonds can benefit from H and/or F surface terminations. Obviously this would not preclude the use of any of these or similar surface termination groups from being used in any combination or any formulation, whether it is water base or oil based. Additionally, surfactants and coupling agents can be added to avoid nanodiamond segregation and/or aggregation.

Nanodiamonds can also be included in a cosmetic, thus creating a cosmeceutical. Thus the collagen enhancing effects can be generated in skin that has been covered with such a cosmetic or cosmeceutical. Non-limiting examples of cosmetics include lipstick, lip gloss, lip liner, liquid foundation, concealer, cream foundation, powder, bronzer, blush (powder, cream, and gel), mascara, eye liner, eye shadow, mineral cosmetics.

In another aspect, the nanodiamond particles can be dispersed in a volatile or evaporatable carrier, such as a water-based carrier. In such cases, the composition can be applied to the skin and the carrier can be evaporated, leaving a nanodiamond residue that can absorb energy and radiate IR to increase the temperature underneath the epidermis.

A variety of ingredients are contemplated for inclusion in the present compositions. Examples of such include without limitation, dispersants, surfactants, stabilizers, carriers, and other ingredients can vary depending on the specific formulation. In addition, a composition can include additives such as fragrance, colorants, vitamin E, herbal supplements, antibiotics, UV absorbers, sun-block agents, and the like. Specific examples of sun-block agents can include TiO₂, CEO₂, ZnO, hBN, talc, clay minerals, and combinations thereof. A more detailed description of various lotions and other types of compositions can be found in U.S. Pat. Nos. 6,207,175 and 6,248,339, which are each incorporated herein by reference in their entireties. Additionally, additives such as antioxidants, moisturizers, collagen sources, chitosan, and the like can be included in the formulations of the present invention.

It is also understood that carriers can vary according to the desired form of the composition. Generally, however, non-limiting examples of cosmetically acceptable carriers include water, gels, glycerin, alcohols, emollients, fatty acids, fatty alcohols, maltodextrin, carrageenans, microcrystalline cellulose (MCC) and other celluloses, sugars, alcohol sugars, lactose, and combinations thereof.

The present compositions can also be formulated so as to be applied with a tissue or wipe. The tissue or wipe composition can include an acceptable carrier and a plurality of nanodiamond particles. Acceptable carriers are known in the art and can include, for example, glycerin, alcohols, water, gels, combinations of these materials, and other known carriers. In addition, such compositions can include additives such as fragrance, colorants, vitamin E, herbal supplements, antibiotics, UV absorbers, sun-block agents, and the like. A more detailed description of facial wipe formulations can be found in U.S. Pat. No. 6,428,794, which is incorporated herein by reference in its entirety. The wipe can thus be used to conveniently apply a sunscreen composition to a subject. In another aspect, the present compositions can be formulated as a spray, including propellants, aerosolized particles, etc. Thus the nanodiamond particles can be formulated with any carrier acceptable in a spray composition.

Nanodiamond particles typically carry an electrical charge, which leads to aggregation and flocculation of particles. In many cases, this aggregation of nanodiamond particles is undesirable. Therefore, an optional dispersant can be included that improves the uniformity of nanodiamond distribution. In this way, a colloidal suspension can be formed in which the nanodiamond particles remain substantially uniformly dispersed over an extended period of time, e.g., typically months or years. It is beneficial for the nanodiamond particles to remain dispersed during the useful shelf-life of the particular composition. The dispersant can be provided in the form of a specific compound separate from the carrier in a liquid nanodiamond composition. However, in certain situations, e.g. for highly viscous compositions, the carrier can also be the dispersant. Thus, in some embodiments such as a solid deodorant, toothpaste, soaps, viscous nail polish, and the like, the carrier can provide sufficient viscous support to prevent agglomeration and/or settling of the nanodiamond particles.

Any suitable dispersant can be used which is compatible with a particular carrier. However, several non-limiting examples of dispersants include anionic surfactants, electrolytes, alcohols, metal chlorides and nitrates of Al, Na, Ca, and Fe, viscous biologically acceptable carriers, and the like. Other suitable nanodiamond dispersants include isopropyl triisosteroyl titanate, polyethylene-oxides, and other anionic surfactants. One specific suitable surfactant which can be used is stearalkonium hectorite. The dispersant can also provide other properties to a composition such as pH control. Further, the amount of dispersant can depend on the amount of nanodiamond present and the viscosity of the composition. However, as a general guideline, the composition can include from 1 wt % to about 30 wt % dispersant.

Nanodiamond particles can be formed using a number of known techniques such as shock wave synthesis, detonation, CVD, and the like. In one aspect, nanodiamond made by explosive detonation can be used. Such nanodiamond particles contain internal defects, voids, microcracks, and in some cases external radicals such as H, N, O, and the like, that can increase the absorbance of UV radiation by the nanodiamond particles. Sp1 and sp2 bonded carbon associated with the nanodiamond particles can also increase the absorption of UV radiation, thus greatly widening the overall absorption bands of the nanodiamond material. A variety of additional nanodiamond formation techniques are also contemplated, including those discussed in U.S. Pat. No. 7,384,436, which is incorporated herein by reference in its entirety.

Various size ranges of nanodiamond particles can be utilized in the present compositions depending on the desired absorption of the composition. Larger nanodiamond particles tend to absorb lower energy radiation. As such, compositions can be formulated to absorb radiation within fairly specific energy ranges by using nanodiamond particles having a size in a specific range. Conversely, compositions can be formulated having a broad range of absorption energies by utilizing a broad range of nanodiamond particle sizes. In one specific aspect, the nanodiamond particles have an average size of from about 5 nm to about 900 nm. In an additional aspect, the average size may be from about 2 nm to about 500 nm. In another specific aspect, the nanodiamond particles have an average size from about 2 nm to about 50 nm. In yet another specific aspect, the nanodiamond particles have an average size of less than or equal to about 10 nm. In a further specific aspect, the nanodiamond particles have an average size from about 50 nm to about 2000 nm.

Furthermore, the concentration of nanodiamond particles can vary depending on the composition and the desired effect. Because of the effectiveness of the present nanodiamond compositions at absorbing electromagnetic radiation, the concentration of nanodiamond particles can be surprisingly low, although such low concentrations are not required. In one aspect the plurality of nanodiamond particles is from about 1 wt % to about 80 wt % of the composition. In another aspect the plurality of nanodiamond particles is from about 1 wt % to about 60 wt % of the composition. In yet another aspect the nanodiamond particles comprise from about 1 wt % to about 20 wt % of the composition. In a further aspect the nanodiamond particles comprise less than or equal to about 5 wt % of the composition. In another aspect, the concentration of nanodiamond particles in the composition can be less than 200 ppm, or less than 100 ppm, or less than 50 ppm of the total composition.

In one aspect of the present invention, some or all of the nanodiamond particles may include a coating, either full or partial, of a non-carbon material. In one aspect, such a non-carbon coating may be an oxide, a nitride, or a boride. In some aspects, the oxide may contain silica. In some aspects of the present invention, nanodiamond particles can be functionalized. Various functional groups are contemplated, and any functional group capable of increasing the absorption of electromagnetic radiation is considered to be within the present scope. In one aspect, for example, a functional group can include amino, carboxyl, hydroxyl, carbonyl, pyridine, and the like, including combinations thereof. In one specific example, the functional group can be and amino/peptide group. Aromatic, hexa-, and penta-cyclic containing compounds are also contemplated, or in general, any ring structure of carbon or boron nitride that can resonate at UV energies. Thus, such functional groups can be used to increase the absorption of UV A, B, and C radiation. In one aspect, the functional group can include benzene, peridine, pyran, thiopyran, and the like. In another aspect, the functional group can include cyclopentane, Pyrrole, furan, thiophene, and the like. Additionally, the nanodiamond particles can be functionalized with graphene and/or hexagonal boron nitride to facilitate UV absorption.

The nanodiamond particles can also be functionalized with a variety of functional groups that can provide skin-related benefits in addition to collagen growth and UV absorption. For example, in one aspect various skin conditions, such as dermatitis, psoriasis, and the like, can be treated using such functional groups. In one aspect, for example, a functionalized group can include nucleic acids, active agents, emollients, moisturizers, and the like. These functional groups, and those discussed above, can be attached to nanodiamond particles directly, or via chemical linker molecules. Additionally, it should be noted that functional groups can perform multiple functionalities, including, for example, skin condition treatment as well as collagen growth enhancement and/or UV protection.

In addition to providing collagen enhancement, introduction of nanodiamond particles into a composition can provide a number of beneficial properties. One of such beneficial properties is an ability of nanodiamonds to absorb oil and other organic materials. Carbon atoms are very small (about 1.5 angstroms); thus, various forms of carbon can pack to form a high atomic concentration. Accordingly, diamond has a high atomic concentration (176 atom/nm³), which contributes to the exceptional hardness of diamond. As a result, any given surface area of a nanodiamond particle can include more atoms than other nanoparticles of the same size.

Although diamond is highly stable, if the nanodiamond surface is free of adsorbent or absorbent, i.e. clean, it is thought that carbon atoms on the surface contain unpaired electrons that are highly reactive. As a result, nanodiamond particles can readily bond to and effectively absorb a variety of atomic species. For example, small atoms such as H, B, C, N, O, and F can be readily adsorbed on the nanodiamond surface, although other atoms can also be absorbed. Hence, nanodiamond particles, with their vast number of surface atoms, can hold a large amount of such adsorbed atoms. For example, nanodiamond particles are capable of absorbing almost as many hydrogen atoms as the number of exposed carbon atoms present on the surface of the nanodiamond material. Thus, nanodiamond particles can be used as storage sites for hydrogen. In addition, those small atoms are building blocks, e.g., H, CO, OH, COOH, N, CN and NO, of organic materials including biological molecules. Consequently, nanodiamond particles can readily attach to amino acids, proteins, cells, DNA, RNA, and other biological materials, and nanodiamond particles can be used to remove skin oils, facial oils, compounds that result in body odor, bacteria, etc.

Thus, nanodiamond particles in a composition can be dispersed on the skin to allow the enhanced growth of collagen. While the nanodiamond particles are associated with the skin, oils and biological matter can be absorbed thereby, to be removed along with the composition.

It is to be understood that the above-described compositions and methods are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in materials, temperature, function, order, and manner of operation, assembly and use may be made without departing from the principles and concepts set forth herein.

What is claimed is:

1. A method of enhancing collagen growth in a subject's skin, comprising:
disposing a composition including a plurality of nanodiamond particles dispersed in a cosmetically acceptable carrier onto a subject's skin;
applying energy to the plurality of nanodiamond particles such that at least a portion of the energy is absorbed by the nanodiamond particles; and
emitting the energy from the nanodiamond particles as IR radiation, wherein the IR radiation is delivered to the skin in order to heat the skin and enhance collagen growth.

2. The method of claim 1, wherein the energy is sunlight.

3. The method of claim 1, wherein the energy is UV radiation.

4. The method of claim 3, wherein the UV radiation is delivered via a UV light source.

5. The method of claim 1, wherein the IR radiation increases temperature underneath epidermal skin by from about 0.5° C. to about 3.0° C.

6. The method of claim 1, wherein the IR radiation increases temperature underneath epidermal skin by from about 1.5° C. to about 2.5° C.

7. The method of claim 1, wherein the composition is formulated as a lotion.

8. The method of claim 1, wherein the nanodiamond particles comprise from about 1 wt % to about 60 wt % of the composition.

9. The method of claim 1, wherein the nanodiamond particles comprise less than or equal to about 5 wt % of the composition.

10. The method of claim 1, wherein the nanodiamond particles have an average size of from about 2 nm to about 500 nm.

11. The method of claim 1, wherein the nanodiamond particles have an average size from about 2 nm to about 50 nm.

12. The method of claim 1, wherein the nanodiamond particles have an average size of less than or equal to about 10 nm.

13. The method of claim 1, wherein the cosmetically acceptable carrier is a member selected from the group consisting of water, gels, glycerin, alcohols, emollients, fatty acids, fatty alcohols, maltodextrin, carrageenans, MCC, sugars, alcohol sugars, lactose, and combinations thereof.

14. The method of claim 1, wherein the cosmetically acceptable carrier is a water-based carrier.

15. The method of claim 1, wherein the cosmetically acceptable carrier further includes a dispersant to disperse the nanodiamond particles.

16. The method of claim 15, wherein the dispersant is a member selected from the group consisting of anionic surfactants, electrolytes, alcohols, metal chlorides, metal nitrates, viscous biologically acceptable carriers, and mixtures thereof.

17. The method of claim 1, wherein the nanodiamond particles include surface functional groups.

18. The method of claim 17, wherein the functional groups are members selected from the group consisting of amino, carboxyl, hydroxyl, carbonyl, pyridine, and combinations thereof.

19. The method of claim 1, wherein the composition includes a sun-block agent that is a member selected from the group consisting of TiO₂, CEO₂, ZnO, and combinations thereof.

20. The method of claim 1, wherein the nanodiamond particles contain at least one of microcracks, voids, and non-bonding carbon to increase energy absorption range of the nanodiamond particles.
